(19) United States
(12) Patent Application Publication
(10) Pub. No.: US 2009/0203800 A1
(43) Pub. Date: Aug. 13, 2009

(54) Cytostatic Composition

(76) Inventors: Sergey Tishkin, Wegberg (DE); Vladislav Nikolaevich Laskavyj, Wegberg (DE)

Correspondence Address: DONN K. HARMS;PATENT & TRADEMARK LAW CENTER, SUITE 100, 12702 VIA CORTINA, DEL MAR, CA, 92014, US

(21) Appl. No.: 12/368,220

(22) Filed: Feb. 9, 2009

Related U.S. Application Data
(60) Provisional application No. 61/065,172, filed on Feb. 9, 2008.

(51) Int. Cl. A61K 31/115; A61P 35/00

(52) U.S. Cl. 514/694

(57) ABSTRACT

A cytostatic composition comprising an effective amount of an aldehyde in a pharmacological salt solution is shown to be effective at inhibiting growth of a number of cancerous cell lines.

The instant application claims the benefit of U.S. Provisional Patent Application 61/065,172, filed Feb. 9, 2008 and incorporated by reference herein in its entirety.

### FIELD OF THE INVENTION

The disclosed device and method relate to the inhibiting of cancer cell growth. More particularly, there is disclosed a cytostatic composition comprising an effective amount of an aldehyde in a pharmacological salt solution which is shown to be effective at inhibiting growth of a number of cancerous cell lines.

### BACKGROUND OF THE INVENTION

Aldehydes have been used previously for preparation of samples from cancerous tissues for microscopic examination and also in some cases as a co-treatment.

For example, PCT Application WO 02/28345 teaches that DNA adducts form when a specific class of chemotherapy agent is administered. Adriamycin is one example given, but ‘class’ is generally referred to elsewhere as an ‘anthracycline or anthracenedione’ and that the formation of these adducts is linked to cytotoxicity and requires the presence of aldehyde. In this application, they describe co-administering the chemotherapy agent with an aldehyde-releasing agent so that the potency of the chemotherapy agent in vivo is increased by the release of additional aldehyde. In some embodiments, the aldehyde is formaldehyde.

U.S. Pat. No. 6,677,309 teaches conjugates of anthracyclines and an aldehyde-releasing agent.

PCT Application WO 2005/120577 teaches conjugates comprising a first moiety that is not a psychoactive drug and a second moiety that is capable of releasing a formaldehyde molecule.

PCT Application WO 2005/034856 teaches a conjugate which has a therapeutic agent bonded to an aldehyde which is ‘protected’ with a ‘chemical trigger’ and may further include a targeting group. In other words, the chemical trigger keeps the conjugate in a prodrug form until it reaches the desired location. In some cases, a targeting molecule is used to direct the conjugate to the desired therapeutic location.

### SUMMARY OF THE INVENTION

According to a first aspect of the invention, there is provided a pharmaceutical composition comprising: an aldehyde suspended in a solution of a pharmaceutically acceptable salt in water. That is, there is provided a pharmaceutical composition comprising: an aldehyde suspended in an aqueous solution of a pharmaceutically acceptable salt.

### BRIEF DESCRIPTION OF THE DRAWINGS

FIG. 1 Pictures of inhibition of colony formation in the large cell lung cancer LXFL 529

FIG. 2 depicts the colony formation of FIG. 1 at a higher power.

FIG. 3 shows a Mean-graph analysis of Cytostatic.

FIG. 4 depicts a Effect of Cytostatic on mouse body weight, following twice daily im dosing at 100 μl/mouse. A: Group median relative body weights over time. B: Individual relative body weights on Day 14

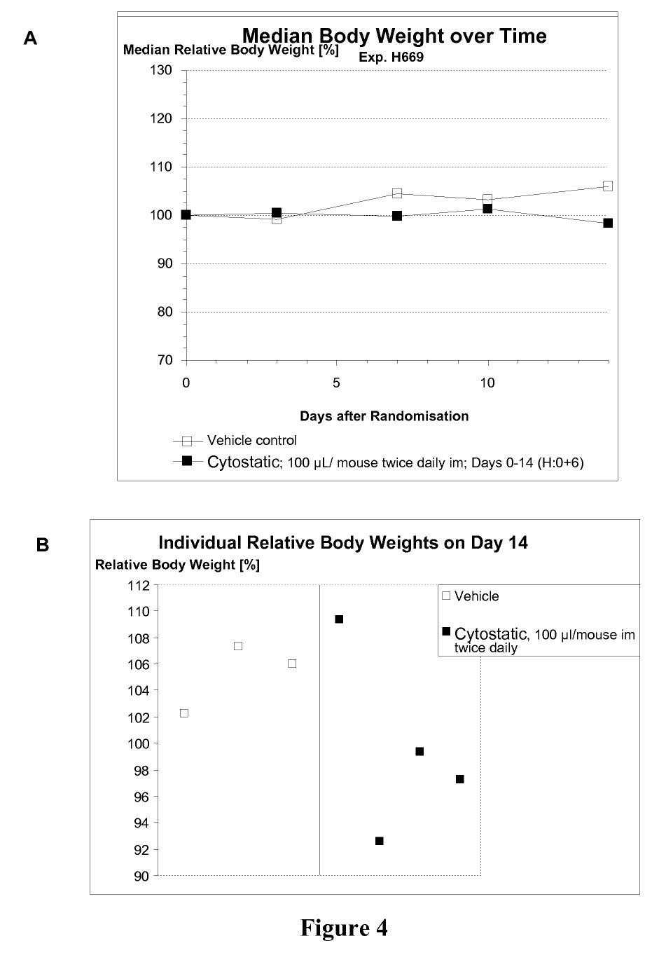

FIG. 5 depicts the concentration effect curves of CC for 4 human tumor cell lines

FIG. 6 depicts the in vitro growth of the cell line MAXF 401NL after 3 days treatment with CC (1st cycle)

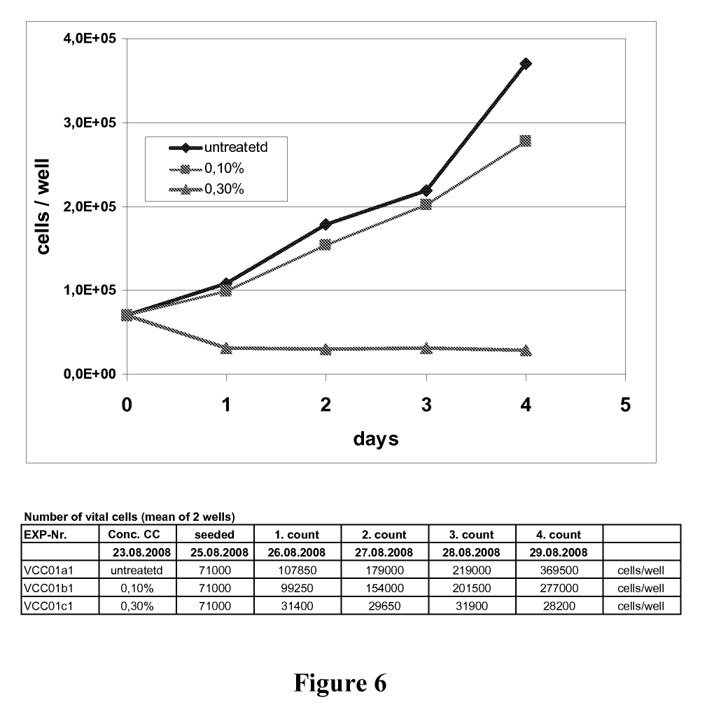

FIG. 7 depicts the in vitro growth of the cell line MAXF 401NL after 3 days treatment with CC (2nd cycle)

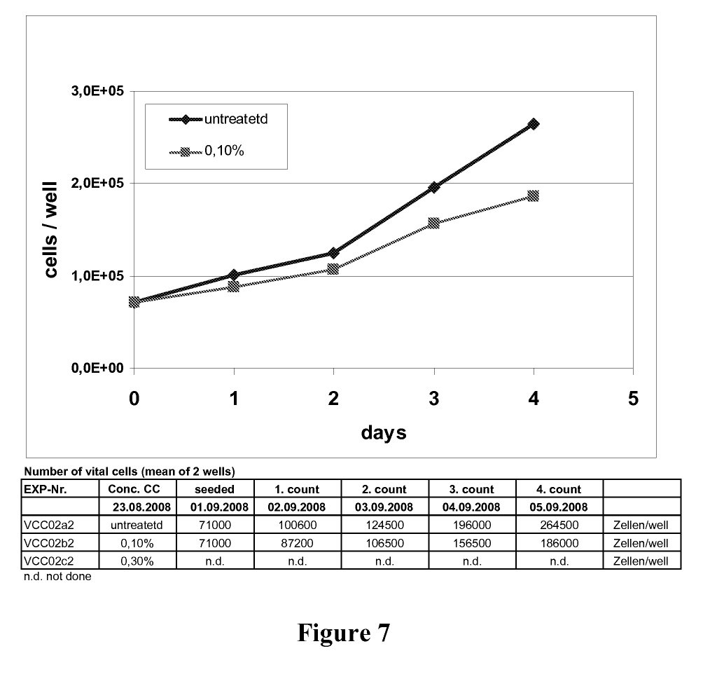

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is an anti-cancer or cytostatic composition comprising an aldehyde suspended in an aqueous salt solution. For convenience, in some instances, the composition is referred to as ‘cytostatic’.

As used herein, an ‘aldehyde’ refers to any organic compound containing a terminal carbonyl group or aldehyde group. In a preferred embodiment, the aldehyde is a natural aldehyde. In yet further preferred embodiments, the natural aldehyde is formaldehyde.

In one embodiment of the invention, a cytostatic composition is prepared by providing a pharmaceutically acceptable salt solution as described below. The formaldehyde is then suspended into the solution at a concentration between 0.00004% to 1.1% alternatively, the final concentration of formaldehyde suspended in the pharmacological saline solution may be between 0.00012% to 0.12%. The cytostatic composition prepared according to this method may be used for treating cancer, as discussed herein.

The concentration of formaldehyde within the composition may be between 0.00004% to 1.1%. Alternatively, the concentration may be between 0.00012% to 0.12%. The maximum concentration of formaldehyde in the composition is 1.1%. The inventors note that above this rate, toxicity causes depression of the internal organs resulting in the composition having little or no effect. For example, injection of higher formaldehyde levels may result in ulceration at the injection site.

As discussed above, the low point of the range is 0.00004% and below this point it is believed that there is no effect.

In a preferred embodiment, the source of the formaldehyde is formalin, for example, medical grade formalin, for example, 40% formalin which comprises 40% formaldehyde mass. As will be appreciated by one of skill in the art, other pharmaceutically acceptable sources of formaldehyde may be used within the invention.

Preferably, the salt solution is a pharmaceutically acceptable or physiological salt solution, for example, a 0.1%-2.0% or 0.1%-1.9% or 0.1%-1.8% or 0.1-1.7% or 0.1%-1.6% or 0.1%-1.5% or 0.1%-1.4% or 0.1%-1.3% or 0.1%-1.2% or 0.1%-1.1% or 0.1%-1.0% or 0.1%-0.9% or 0.2%-2.0% or 0.3%-2.0% or 0.4%-2.0% or 0.5%-2.0% or 0.6%-2.0% or 0.7%-2.0% or 0.8%-2.0% or 0.9%-2.0% or 0.5-1.5% or 0.5%-1.3% or 0.6-1.4% or 0.6%-1.2% or 0.7%-1.3% or 0.7%-1.1% or 0.8%-1.2% or 0.8%-1.0% or a 0.9% sodium chloride solution or Ringer's solution or saline solution. Preferably, the physiological salt solution is at or near the physiological pH of the individual to which it is to be administered, that is, the pH of the pharmaceutically acceptable or physiological salt solution is between 7.2 and 7.6.

In a preferred embodiment, there is provided a cytostatic composition comprising 0.00004% to 1.1% formaldehyde suspended in a physiological salt solution. In a preferred embodiment, the concentration of the formaldehyde in the physiological salt solution is between 0.00012% to 0.12% (v/v). In some embodiments, the physiological salt solution is an NaCl solution, a saline solution or Ringer's solution. In some embodiments, the salt solution is 0.1-2.0% or 0.9% sodium chloride for injections.

In one embodiment of the invention, a cytostatic composition is prepared by providing a 0.9% solution of sodium chloride in water. The formaldehyde is then suspended into the solution at a concentration between 0.00004% to 1.1% alternatively, the final concentration of formaldehyde suspended in the pharmacological saline solution may be between 0.00012% to 0.12%. The cytostatic composition may be used for treating cancer, as discussed herein.

As discussed below, the cytostatic composition is believed to be effective at ‘converting’ cells undergoing anaerobic respiration to aerobic respiration which in turn reduces or inhibits proliferation or the growth rate of cancerous cells but is expected to have little or no effect on cells already undergoing aerobic respiration.

While not wishing to be bound by a specific theory, the inventors believe that the formaldehyde when mixed with pharmaceutically acceptable aqueous salt solution, for example but by no means limited to 0.9% NaCl aqueous solution undergoes a transition which enables the composition to penetrate the metabolism within the cell structure in the body thereby releasing bound formaldehydes within the tumor and causing growth of the tumor to cease and then diminish the tumor itself, that is, diminishing the size of the tumor itself.

It is noted that Otto Warburg previously found that cancerous cells typically use anaerobic glucose respiration which involves the formation of lactic acid as the nutritious substance. According to Warburg, ‘regeneration’ of cells into cancerous ones using anaerobic respiration leads to autonomous existence of cells. Regeneration of cells causes precancerous changes in tissues, for example, but by no means limited to changes in acidity, energy consumption, breathing, etc. Thus, one stage of the transformation of a normal or pre-cancerous cell to a cancerous cell is the dedication of the cell to anaerobic glucose respiration.

Biological respiration in cells progresses by two phases. The first phase is the anaerobic one (oxygen free) and the second one is the aerobic one (with oxygen). Glycolysis (anaerobic phase of respiration) ends up with turning pyruvic acid into lactic acid. The anaerobic phase of respiration provides only two ATP molecules against one glucose molecule. The second phase of biological respiration (aerobic) results in the synthesis of 38 ATP molecules against one glucose molecule. Thus, oxygen breathing organisms use energy of carbohydrates 19 times more effectively than anaerobes.

As an alternative to existing methods of treatment of oncology conditions such as for example increasing immunity, oxygenation of the organism, hyperthermia, photodynamic therapy, vascular blockage of tumor, traditional therapies, radial therapy, creation of special proteins, blocking oncogenes, use of nanotechnologies and the like, the inventors believe that another approach to the treatment of oncologic conditions may be changing the metabolism of oncocells or cancerous cells from anaerobic respiration to aerobic respiration.

In view of this, the inventors used formaldehyde, one of the natural metabolites which is contained in minimal quantities in all organs, tissues and liquid mediums. Formaldehyde is typically made within cells as a result of metabolic processes and it is easily inactivated by enzyme systems (L. V. Miretskaya, P. Ya. Shvartsman. Cytology.-1982.-Volume XXIV.-No 9.-page 1059). In particular, formaldehyde stimulates synthesis of hexulose-phosphate synthase, which is a key enzyme of the ribulose mono-phosphate cycle. Furthermore, the mono carbon radical of formaldehyde gets actively involved in the biosynthesis of various compounds. Formaldehyde also has immune modulating and antiviral properties.

In view of this, as discussed herein, the inventors have researched the effects of formaldehyde-containing agents and/or compositions on metabolic processes in cells.

For example, injection of a cytostatic composition as described herein into rabbits at an effective dosage significantly increases enzyme activity associated with amino acid metabolism, for example, aspartate aminotransferase and alanine aminotransferase, in animal organisms. This in turn leads to lower utilisation of alanine and asparagine in the synthesis of proteins, which is demonstrated by a reduction of protein level in the blood serum of the subject.

For example, an increase of alanine aminotransferase activity leads to an increase in pyruvic acid level, from which acetyl-CoA is created by way of oxidating decarboxylation in the mitochondria. A significant quantity of acetyl-CoA gets “burnt out” in the cycle of di- and tri-carbon acids making large quantity of hydrogenated nicotinamide-adenine dinucleotides and flavin adenine dinucleotides, which are used in the mitochondria for the oxidation-related synthesis of ATP.

Also, an increase in creatine phosphakinase activity points to an increase in the energy provision of cells. At the same time, a reduction of lactate dehydrogenase activity occurs, which points to predomination of aerobic processes of respiration over anaerobic ones. This means that the main mass of pyruvic acid, formed in glycolysis, is subjected to oxidative decarboxylation and outputs a large quantity of acetyl-CoA, which is used in the aerobic phase of oxidation of organic elements in cells. Excess acetyl-CoA is used for synthesis of various lipoids, in particular for cholesterol synthesis, which can be detected as a significant increase of general cholesterol level in the subject's blood.

Thus, it is believed that the cytostatic composition described herein activates aerobic respiration in cells, which in turn explains its cytostatic effect on various lines of tumor cells. That is, as discussed above, cancerous cells typically undergo anaerobic respiration while normal or non-cancerous cells undergo aerobic respiration. However, administration of an effective amount of the cytostatic compound converts these cells from anaerobic respiration to aerobic respiration. Furthermore, the cytostatic composition is expected to have little or no effect on normal cells undergoing aerobic respiration, as discussed herein.

Alternatively, the inventors note that formaldehyde easily reacts with free lysine and arginine amidogen and during this process, the carbonyl group turns into an oxy group and the amidogen turns into imino group. Imino group hydrogen and hydroxy oxygen as well as hydroxyl group hydrogen and imino group nitrogen can create an intramolecular hydrogen bond. Meanwhile, imine (Schiff's base) is created through a methylcarbinolamine intermediate phase:

HCOH+NH₂—CH(R)—CO⁻→H₂C(OH)—NH—CH(R)—CO⁻→HCH═N—CH(R)—CO⁻+H₂O

Where NH₂—CH(R)—CO is a fragment of protein molecule.

Binding of free amidogens leads to their loss of ability to accept ions of hydrogen. Concentration of free ions of hydrogen in intracellular content gets somewhat increased, that is, the pH is shifted to acidic direction. Because the optimum pH of most glycolysis enzymes occurs in an alkaline environment, the proportion of oxybiotic oxidation of carbohydrates gets increased.

In addition, the subsequent protonation of imino groups creates conditions for the initiation of hydrogen bonds. Amidogen as part of lysine amino acid chemical group interacts much the same. In the opinion of the inventors, free arginine amidogen does not differ from N-end protein amidogens and radical lysine amidogen in its functional activity and has a homotypic reaction. Such interaction leads to changes in the conformation of protein molecules and in doing so, to changes in their physical/chemical properties. Proteines-histones which are part of chromosomes as nucleoprotein contain large numbers of diaminomonocarboxylic acids of lysine and arginine. Formation of hydrogen bonds following the reaction of addition of formaldehyde blocks intermolecular van der Waals interaction between carboxylic DNA groups and free amidogens of histones. As a result, former transcription zones may disappear and new ones appear (formation of RNA-copies of genes), which lead to changes in quantity and quality of cell proteins. In summary, there is a possibility to achieve phenotypic mutation under unchanged genotype.

As used herein, an ‘effective amount’ is an amount of the cytostatic composition that is sufficient to accomplish one or more of the following: increase aerobic respiration within a population of cancerous cells, for example, a tumor, compared to an untreated or control or mock treated population of cells of similar age and condition; reduce or inhibit growth rate or proliferation of a population of cancerous cells, for example, a tumor, compared to an untreated or control or mock treated population of cells of similar age and condition; reduce tumor volume growth rate compared to an untreated or control or mock treated population of cells of similar age and condition; result in a longer period of remission compared to an untreated or control or mock treated population of cells of similar age and condition; and reduce the severity of one or more of the symptoms associated with the cancer compared to an untreated or control or mock treated population of cells of similar age and condition.

As referred in the examples shown below, cytostatic composition of the invention has been shown to be effective in vitro tests for large cell lung cancer, renal cancer, colon cancer, bladder cancer, gastric cancer, head and neck cancer, liver cancer, adeno lung cancer, small cell lung cancer, mammary cancer, ovary cancer, pancreatic cancer, prostate cancer and as well as melanoma, pleuramesothelioma, and sarcoma. Accordingly and as discussed below, the cytostatic composition of the instant invention has been shown to be suitable treatment for a wide variety of cancer types and could be used as a treatment for any disease or disorder characterized by anaerobic respirative growth of a population of cells.

Thus, the cytostatic composition of the invention may be used to treat or prevent cancer or a cancerous growth in an individual in need of such treatment, that is, an individual diagnosed with cancer, suspected of having cancer or at risk of developing cancer. As discussed herein, suitable cancers include but are by no means limited to large cell lung cancer, renal cancer, colon cancer, bladder cancer, gastric cancer, head and neck cancer, liver cancer, adeno lung cancer, small cell lung cancer, mammary cancer, ovary cancer, pancreatic cancer, prostate cancer and as well as melanoma, pleuramesothelioma, and sarcoma. Accordingly and as discussed below, the cytostatic composition of the instant invention has been shown to be suitable treatment for a wide variety of cancer types and can be used as a treatment for any disease or disorder characterized by anaerobic respirative growth of a population of cells.

The invention will now be further illustrated by way of examples. However, the invention is not necessarily limited by the examples.

### ABBREVIATIONS

Abbreviations used herein include: body weight loss (BWL), cyclin dependent kinase (CDK), carbon dioxide (CO₂), day(s) (d), dimethyl sulfoxide (DMSO), fetal calf serum (FCS), 5-fluoruracil (5-FU), gram (gm), immunohistochemistry (IHC), inhibitory concentration where a T/C-value=100−x is reached (ICₓ), intramuscular (im), immune modulatory compound (test compound of the study, that is, the cytostatic compound) (CC), Iscove's Modified Dulbecco's Medium (IMDM), infiltrative (inf.), kilogram (kg), liter (L), milligram (mg), milliliter (mL), moderately differentiated (md), milligram (mg), microgram (μg), milliliter (ml), microliter (μl), micrometer (μm), not available (n.a.), not determined (n.d.), Naval Medical Research Institute, USA (NMRI), non small cell (NSC), papillary (pap), phosphate buffered saline (PBS), poorly differentiated (pd), Roosevelt Park Memorial Institute (RPMI), test versus control value (T/C-value), unit (U), undifferentiated (ud), volume per volume (v/v), well differentiated (wd), without (w/o), weight per volume (w/v).

### Example 1

The antitumor efficacy of cytostatic composition as described above was evaluated in 27 human tumor xenografts in vitro using a clonogenic assay. The tumor test panel comprised 1 to 4 models of 15 different human tumor types, which were bladder cancer, colon, gastric, head and neck, liver, non small cell lung (adeno and large cell), small cell lung, mammary, ovary, pancreatic, prostate and renal cancer as well as melanoma, pleuramesothelioma, and sarcoma. The cytostatic composition was studied at 6 concentrations ranging from 0.001% to 100.0%. Antitumor effects were recorded as inhibition of colony formation in relation to untreated controls (T/C-values).

The cytostatic composition also referred to herein as ‘cytostatic’, inhibited tumor colony formation in a concentration-dependent manner. The mean IC70-value was determined with 0.462%, the mean IC50-value was determined with 0.195%. Above average activity of Cytostatic was seen against tumor models of large cell lung cancer (LXFL 529), small cell lung cancer (LXFS 615, LXFS 650), mammary cancer (MAXF 401), melanoma (MEXF 989), and prostate cancer (PRXF MRIH1579). The most sensitive tumors were the small cell lung cancer LXFS 650 and the melanoma MEXF 989. The IC70-values in these tumor models were more than 100-fold lower compared to the mean IC70-value.

### Objective

In the present study, Cytostatic was investigated for anticancer activity in vitro in 27 tumor xenografts. A clonogenic assay was used in order to investigate possible tumor type selectivity.

In the clonogenic assay (=tumor colony assay, TCA), inhibition of colony formation of tumor stem cells growing in soft agar is examined. Tumor stem cells, which are responsible for growth, the metastatic and infiltrative potential of a tumor, are prepared directly from human tumor xenografts growing in nude mice. Hence, the clonogenic assay reflects better the in vivo situation than in vitro assays using permanent tumor cell lines and has been found to be a highly predictive test for further in vivo evaluation of anticancer drugs.

### Vehicles and Concentrations

Cytostatic was tested at 6 concentrations. The highest concentration tested was 0.004%, as shown below, which was the maximum allowed concentration of the vehicle in the test set as 100% of Cytostatic. IMDM supplemented with 10% v/v saline solution was also used as a control vehicle.

| Relevant concentrations | Formaldehyde concentration in |
|---|---|
| as indicated in the tests | Cytostatic |
| 100% | 0.004% |
| 10% | 0.0004% |
| 1% | 0.00004% |
| 0.1% | 4 × 10⁻⁶ |
| 0.01% | 4 × 10⁻⁷ |
| 0.001% | 4 × 10⁻⁸ |

### Tumor Models

The origin of the xenografts has been described previously (Berger et al, 1990, Ann. Oncol. 1: 333-341; Scholz et al., 1990, Eur. J. Cancer 26: 901-905; Fiebig et al, Eur. J. Cancer 40: 802-820). Cytostatic was tested in a total of 27 human tumor xenografts. The tumor test panel comprised 1 to 4 models of 15 different human tumor types, which were bladder cancer, colon, gastric, head and neck, liver, non small cell lung (adeno and large cell), small cell lung, mammary, ovary, pancreatic, prostate and renal cancer as well as melanoma, pleuramesothelioma, and sarcoma.

### Tumor-Colony-Assay

Preparation of Single Cell Suspensions from Human Tumor Xenografts

Solid human tumor xenografts growing subcutaneously in serial passages in thymus aplastic nude mice (NMRI nu/nu strain,) were removed under sterile conditions, mechanically disaggregated and subsequently incubated with an enzyme cocktail consisting of collagenase type IV (41 U/ml) (Sigma), DNase I (125 U/ml) (Roche), hyaluronidase type III (100 U/ml) (Sigma) and dispase II (1.0 U/ml) (Roche) in RPMI 1640-Medium (Life Technologies) at 37° C. for 45 minutes. Cells were passed through sieves of 200 μm and 50 μm mesh size and washed twice with sterile PBS-buffer. The percentage of viable cells was determined in a Neubauer-hemocytometer using trypan blue exclusion.

Culture Methods of Cells from Human Tumor Xenografts

The clonogenic assay was performed in a 24-well format according to a modified two-layer soft agar assay introduced by Hamburger & Salmon. The bottom layer consisted of 0.2 ml/well IMDM (Life Technologies) (supplemented with 20% (v/v) fetal calf serum (Sigma), 0.01% (w/v) gentamicin (Life Technologies) and 0.75% (w/v) agar). 2×10⁴ to 4×10⁴ cells were added to 0.2 ml of the same culture medium supplemented with 0.4% (w/v) agar and plated in 24-multiwell dishes onto the bottom layer. The test compound was applied by continuous exposure (drug overlay) in 0.2 ml culture medium. Every dish included six untreated control wells and drug-treated groups in triplicate at 6 concentrations. Cultures were incubated at 37° C. and 7.5% CO₂ in a humidified atmosphere for 6-18 days and monitored closely for colony growth using an inverted microscope. Within this period, in vitro tumor growth led to the formation of colonies with a diameter of >50 μm. At the time of maximum colony formation, counts were performed with an automatic image analysis system (OMNICON 3600, Biosys GmbH). 24 hours prior to evaluation, vital colonies were stained with a sterile aqueous solution of 2-(4-iodophenyl)-3-(4-nitrophenyl)-5-phenyltetrazolium chloride (1 mg/ml, 100 μl/well) (Sigma).

### Data Evaluation

An assay was considered fully evaluable, if the following quality control criteria were fulfilled:
mean number of colonies in the control wells of 24-multiwell plates≧20 colonies with a colony diameter of >50 μm
coefficient of variation in the control wells of each plate≦50%
the positive reference compound 5-fluorouracil (5-FU, at the cytotoxic concentration of 1.0 mg/ml) must effect a reduction of colony number to <30% of the controls
or initial plate counts on days 0 or 2 must be <20% of the final control count.

Drug effects were expressed by the percentage of colony formation, obtained by comparison of the mean number of colonies in the treated wells with the mean colony count of the untreated controls (relative colony count expressed by the test-versus-control value, T/C-value [%]):

$$\frac{T}{C} = \frac{colony {count}_{treated group}}{colony {count}_{control group}} \cdot 100 [ \% ] .$$

IC₅₀- and IC₇₀-values, being the drug concentrations necessary to inhibit colony formation by 50% (T/C=50%) and 70% (T/C=30%), respectively, were determined by plotting compound concentration versus relative colony count. Mean IC₅₀- and IC₇₀-values were calculated according to the formula

$$mean {IC}_{50 , 70} = {10}^{( \frac{{\sum }_{x = 1}^{n} log ( {IC}_{50 , 70} )}{n} )}$$

with x the specific tumor model, and n the total number of tumor models studied. If an IC₅₀- or IC₇₀-value could not be determined within the examined dose range (because a compound was either too active or lacked activity), the lowest or highest concentration studied was used for the calculation.

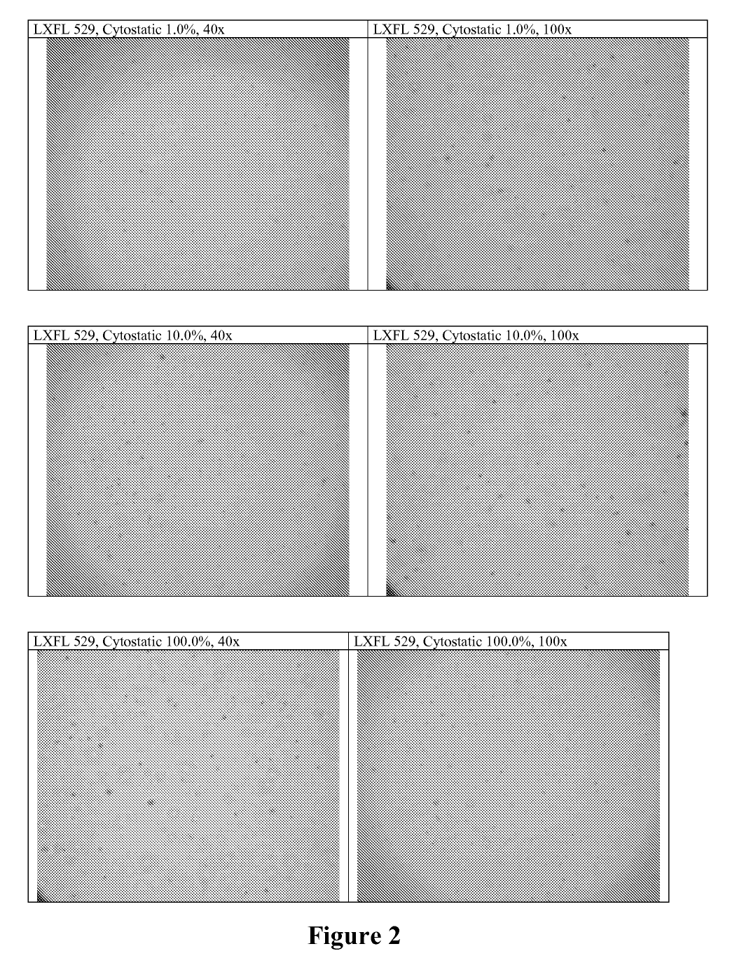

In the mean graph analysis the distribution of IC50-(IC70-) values obtained for a test compound in the individual tumor types is given in relation to the mean IC50-(IC70-) value, obtained for all tumors tested (shown in FIG. 2). The individual IC50-(IC70-) values are expressed as bars on a logarithmically scaled axis. Bars to the left demonstrate IC50-(IC70-) values lower than the mean value (indicating more sensitive tumor models), bars to the right demonstrate higher values (indicating rather resistant tumor models). The mean graph analysis therefore represents a fingerprint of the antiproliferative efficacy of a compound.

### Results

The ability of Cytostatic to inhibit the growth of tumor stem cells to colonies was examined in 27 cell suspensions derived from solid human tumor xenografts of various tumor types. The cell preparations formed 123 to 860 colonies in the untreated control wells within 6 to 20 days, depending on the cell type as described in Table 1.

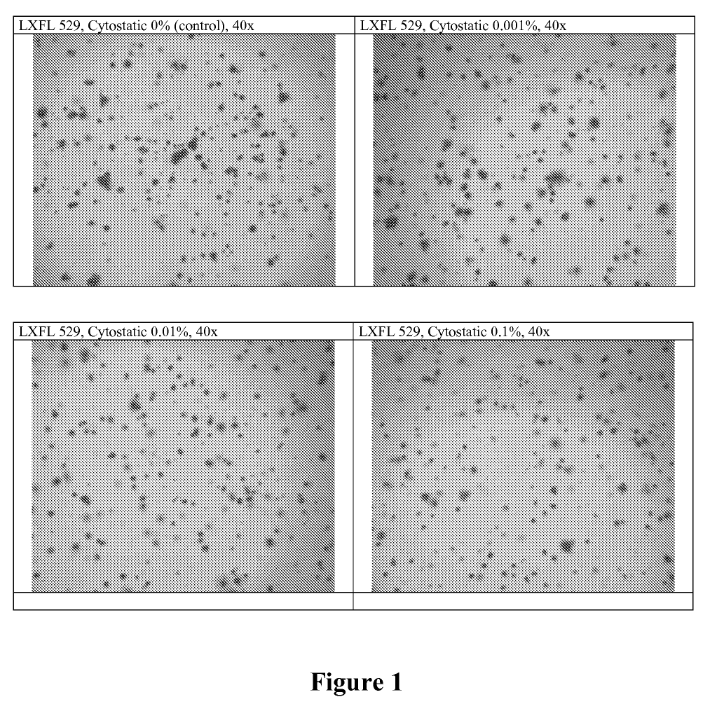

The data outcome of the untreated controls were within the expected range. 5-Fluorouracil which was used as a positive control of growth inhibition showed good antitumor activity. The results of the in vitro testing of Cytostatic are summarized in Table 2 and FIG. 1. Table 2 shows the overall in vitro response rate, i.e. the growth inhibiting activity of Cytostatic at each test concentration. Antitumor activity was defined as inhibition of colony formation to <30% of the untreated controls.

Concentration-dependent inhibition of tumor colony formation was observed. Concentration-response relationships were partly very steep in the range between 0.1 and 1.0%. Cytostatic effected inhibition of colony formation by more than 70% in 1 out of 27 tumor models (4%) at a concentration of 0.001%. At 0.01% and 0.1% Cytostatic was active in 2/27 tumors (7%), at 1.0% in 23/27 tumors (85%), at 10.0% in 25/27 tumors (93%), and at 100.0% in 27/27 tumors (100%) (Table 3). The mean IC₅₀ was determined with 0.195%, the mean IC₇₀ was determined with 0.462% (FIG. 2).

The antitumor selectivity profile of Cytostatic was obtained from mean graph analysis (FIG. 2). The most sensitive tumors in the present study were the small cell lung cancer LXFS 650 (IC₇₀<0.001%) and the melanoma MEXF 989 (IC₇₀=0.004%). Above average activity of Cytostatic was in addition seen against the large cell lung cancer LXFL 529 (IC₇₀=0.162%), the small cell lung cancer LXFS 615 (IC₇₀=0.31%), the mammary cancer MAXF 401 (IC₇₀=0.243%), and the prostate cancer PRXF MRIH1579 (IC₇₀=0.234%) (FIG. 1). Thus, overall antitumor efficacy was observed in 2 out of 2 small cell lung cancers, 1/3 melanomas, 1/3 mammary cancers, 1/4 non small cell lung cancers, and 1/2 prostate cancers.

### Discussion and Conclusions

In the present study, Cytostatic was characterized for its ability to inhibit the in vitro growth of tumor stem cells to colonies with a diameter of more than 50 μm.

Overall, the compound showed activity in a variety of different tumors, as evident from the concentration-dependent inhibition of colony formation in these tumors. Selectivity for individual tumors was well pronounced. The IC₇₀-values in the most sensitive tumor models were more than 100-fold lower compared to the mean IC₇₀-value.

### Example 2

### Evaluation of the Tolerability of Cytostatic Composition (Cytostatic) in Tumor-Free Nude Mice

The tolerability of Cytostatic Composition (Cytostatic) was investigated in male NMRI nu/nu mice, following twice daily i. m. dosing at 100 μl/mouse for 2 weeks. Mortalities and body weight changes were recorded and compared with corresponding data obtained for vehicle control mice that received a 0.9% NaCl solution. The group sizes were 4 Cytostatic-treated and 3 vehicle control mice. After 2 weeks mice were necropsied and subjected to a blood cell count.

Cytostatic was very well tolerated. There were no mortalities, and the maximum median body weight loss was 1.7% recorded on Day 14. At that point the median relative body weight of vehicle control mice had increased by 6%. Necropsy and blood cell analysis did not reveal any gross abnormalities.

In conclusion, no severe adverse effects are to be expected with Cytostatic given twice daily i. m. at 100 μl/mouse.

The study objective was to analyse the tolerability of Cytostatic in tumor-free NMRI nu/nu mice, following dosing at 100 μl/mouse twice daily im. This study included: evaluation of tolerability determined as mortality and body weight loss; necropsy at termination; and analysis of blood cells at the end of the dosing period.

Animal Information
Specific Information

| | |
|---|---|
| Mouse strain: | NMRI nu/nu |
| Total number of mice randomized | 7 males |
| Body weight range at randomization | 26.6-32.8 g |
| Approximate age at randomization: | 4-6 weeks |

### Animal Health

All experiments were conducted according to the guidelines of the German Animal Health and Welfare Act (Tierschutzgesetz).

Animal health was examined prior to randomization to ensure that only animals of good health were selected to enter testing procedures.

### Experiments; Grouping and Randomization of Animals

This study consisted of one experiment comprising a test group receiving Cytostatic and a vehicle control group. The group size was either 4 (test group) or 3 mice (vehicle control group). Mice were dosed for 15 consecutive days and sacrificed one day after administration of the final dose. During the observation period mice were monitored for mortalities and clinical signs and weighed twice weekly. At termination mice were necropsied and blood samples (for blood cell analysis) and organ samples (for fixation) were collected. Blood cell analysis was conducted at Vetmedlab. Blood cell analysis was carried out in Week 29. An overview over the randomization data is given in Table 3, below. The day of randomization was designated as Day 0. Day 0 was also the first day of dosing.

### Animal Identification

Animals were arbitrarily numbered using ear clips. At the beginning of the experiments, each cage was labelled with a record card, indicating the experiment number, date of randomization, mouse strain, gender, and individual mouse number. After randomization group identity, test compound, dosage, schedule, and route of administration were added.

### Housing Conditions

### Husbandry

The animals were housed in Tecniplast R individually ventilated cages. According to group size the animals were housed either in MacrolonM type III cages (maximum 8 mice/cage) or type II long cages (maximum 5 mice/cage). The cages were sterilized at 121° C. before use and changed twice a week. The temperature inside the cages was maintained at 25±1° C. and relative humidity at 60±10%. The animals were kept under a natural daylight cycle.

### Diet and Water Supply

The animals were fed Altromin Extrudat 1439 Rat/Mouse diet. The diet was purchased from Altromin GmbH (Lage, Germany).

Water was sterilized at 121° C. for 30 minutes. After sterilization 0.9 g/l potassium sorbate was added, the pH was adjusted to 2 with 1 N HCl. Water consumption was visually monitored daily, the bottles were changed twice a week. Food and water were provided ad libitum.

### Bedding

The dust free animal bedding Lignocel FS 14 produced by Rettenmaier & Söhne Faserstoffwerke (Ellwangen-Holzmühle, Germany) was purchased from ssniff Spezialdiäten GmbH (Soest, Germany). The bedding was renewed twice a week.

The producer analyzes the dust-free bedding every 3 months with respect to biological/fungal contamination and content of phosphate esters, arsenic, cadmium, lead and mercury. These analyses are carried out at the Agriculture Analyses and Research Institute, Ministry of Agriculture, Kiel, Germany. The quality certificates are deposited at Rettenmaier & Söhne Faserstoffverke (Ellwangen-Holzmühle, Germany).

Treatment Procedure

Route of Administration

All treatments were given i. m.

### Drug Dosage and Treatment Regimen

Cytostatic at concentration of 0.12% of formaldehyde and the vehicle were given at 100 μL/mouse twice daily. From Monday to Friday the time interval between the 2 daily doses was approximately 6 h. On Saturday and Sunday this time interval was shorter. One of the 2 daily doses was injected into the right flank and the other one into the left flank.

Observations

### Mortality

Mortality checks were conducted daily.

### Body Weight

Mice were weighed twice a week. Relative body weights of individual mice were calculated by dividing the individual body weight on Day X (BWₓ) by the individual body weight on Day 0 (BW₀) multiplied by 100%.

$$Ind . Relative Body Weight ( Dayx ) = \frac{B {W}_{x}}{B {W}_{0}} \times 100 \%$$

Group median relative body weights were calculated as well, considering only the weights of mice that were alive on the day in question.

### Termination Procedures, Necropsy and Collection of Blood Samples

On Day 15, ie. one day after the final day of dosing, blood was collected into EDTA tubes by sublingual bleeding. In addition, two blood smears per mouse were prepared by drawing out a drop of blood on a microscopic slide.

Subsequently, mice were sacrificed by cervical dislocation and necropsied according to standard protocols. Organs were collected in 10% buffered formalin for <24 hours and then transferred to, and stored in, 70% ethanol. For analysis, blood and blood smears were shipped to Vet Med Labor GmbH, Moericke-strasse 28/3, D-71636 Ludwigsburg (Germany) at ambient temperature (<20° C.) on the same day. Blood cell counts were then performed one day later.

### RESULTS AND DISCUSSION

### Mortality and Body Weight Change

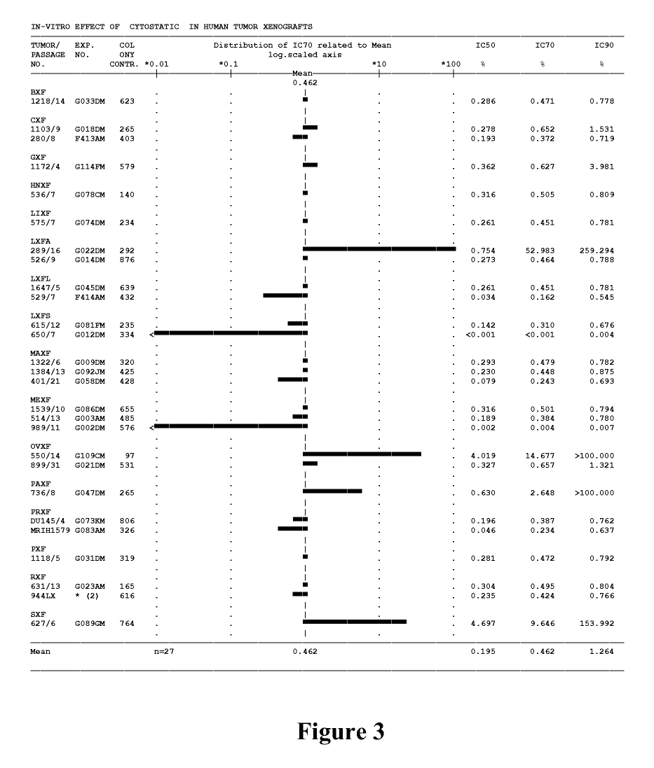

Results are summarized in Table 4 and in FIG. 3.

Treatment with Cytostatic was very well tolerated. All Cytostatic-treated mice survived as did all vehicle control mice. The maximum median body weight loss was minimal (1.7% recorded on Day 14). For comparison, the maximum median body weight loss observed for the vehicle control group was 0.7% (Day 3). On Day 14, i.e. at the end of the 2-week-dosing period, 1 out of 4 Cytostatic-treated mice had gained weight (mouse# 6946, weight gain approximately 9.5%) while the 3 remaining mice had lost between 1 and 7.5% of their initial weight. For comparison, at that point the 3 vehicle control mice had gained between 2.5 and 7.5% of the initial body weight. Because of the small group sizes these differences in body weight changes were statistically not significant (p>0.05, two-sided U-rank test by Mann-Whitney-Wilcoxon). It should also be noted that, following twice daily im dosing of Cytostatic for two weeks, no inflammations developed at the injection sites.

### Necropsy and Blood Cell Analysis

Results are summarized in Tables 4 and 5. Macroscopic inspection of all major organs at termination after 2 weeks of twice daily Cytostatic treatment did not reveal any consistent abnormalities, except that 3 out of 4 Cytostatic-treated but none out of 3 vehicle control mice were rated as adipose. Similarly, no gross abnormalities were detected following analysis of blood cells. Because of the small number of analyzed samples a possible Cytostatic-induced increase of the number of segmented neutrophils and a possible decrease of the number of lymphocytes need to be confirmed in an independent experiment. At this point, the available blood cell counts suggest that Cytostatic had no obvious impact on the immune status of treated mice.

### CONCLUSION

Cytostatic given i. m. at 100 μl per mouse twice daily was very well tolerated. No adverse effects are to be expected with Cytostatic given at this dose level.

### Example 3

### Evaluation of the Antitumor Activity of an Cytostatic Compound (CC) in Human Tumor Cell Lines

The antitumor activity of CC was determined using 4 human tumor cell lines of the proprietary cell line panel (LXF 529L, MAXF 401NL, LXFA 289L, OVXF 899L) in monolayer proliferation and cytoxicity assay.

As shown in Table 6, the mammary cancer cell line MAXF 401 NL was found to be the most sensitive cell line, exhibiting an IC50 value of 0.127% (v/v).

In a second step, MAXF 401NL cells were continuously treated for 3 days with 0.3% CC (more cytotoxic concentration) and 0.1% CC (subtoxic concentration). After treatment, cells were washed twice with PBS and seeded at a cell density of 71.000 cells/well in 24 well cell culture plates (without CC). Cells were counted daily to investigate the effect of pre-treatment with CC on growth rate of the cell line (1st cycle of growth kinetics). In parallel pre-treated cells were passaged under standard cell culture conditions and after 1 week growth rates were determined in a 2nd cycle.

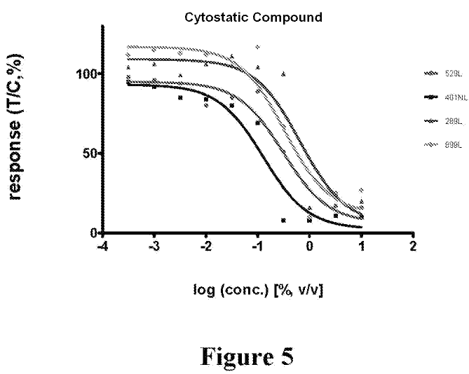

As is apparent from FIG. 5, 0.1% pre-treatment with CC resulted in a slightly reduced growth rate of the cell line MAXF 401NL. After 4 days, the number of vital cells in the untreated group increased from 71.000 up to 369.500 cells, in the 0.1% pre-treated group from 71.000 up to 277.000 cells (25% reduction). In the 0.3% pre-treated group a clear reduction of vital cells was found. However, most of the cells did not attach to the bottom of the plate after seeding, presumably due the advanced damage after the previous treatment with 0.3% CC. Furthermore, passaging this group was not possible, because the cells did not anymore attach to bottom of the plate. Thus, the 0.3% CC group was not available for the 2^{nd} cycle of the growth kinetics. Interestingly, similar to the 1^{st} cycle of cell count, in the 2^{nd} cycle a 30% reduction of cell growth was found after 4 days (264,500 cells in the untreated control group vs 186,000 cells in the 0.1% treated group).

In conclusion, CC showed concentration-dependent activity in the 4 human tumor cell lines as tested with IC₅₀ values in the range from 0.127% (v/v) to 0.657% (v/v).

Investigating the mammary cancer cell line MAXF 401NL indicated slight reduction of the cell growth after pre-treatment with 0.1% CC.

Tests on MAXF 401NL cells pre-treated with the cytostatic compound show slightly reduced growth rate: 25% reduction in first passage and 30% reduction in second passage compare to untreated control. This is evidence of metabolism changes in cancer cells initially induced by the cytostatic compound and apparent transformation of those cells into normal cell condition.

(non-oncogenic).

Thus, as discussed above, the cytostatic composition transforms cancerous cells to non-cancerous/normal cells possibly by changing metabolism—through the movement of the balance of glucose oxidation/degradation from anaerobic to aerobic.

While the preferred embodiments of the invention have been described above, it will be recognized and understood that various modifications may be made therein, and the appended claims are intended to cover all such modifications which may fall within the spirit and scope of the invention.

TABLE 1
Human tumor xenografts examined in the clonogenic assay.

| | | | Incubation | | 5-FU |
|---|---|---|---|---|---|
| | | | time | Colony | control, |
| Tumor type | Tumor no. | Histology | [days]^{a} | number^{a} | T/C [%]^{b)} |
| Bladder | BXF 1218 | transitional cell carcinoma | 6 | 653 | 2 +++ |
| Colon | CXF 1103 | adeno carcinoma, pd | 20 | 280 | 10 +++ |
| | CXF 280 | carcinoma, ud | 19 | 458 | 21 ++ |
| Stomach | GXF 1172 | signet-ring cell carcinoma, pd | 11 | 598 | 15 ++ |
| Head & neck | HNXF 536 | squamous epithelium carcinoma, | 20 | 229 | 9 +++ |
| | | wd | | | |
| Liver | LIXF 575 | hepatocellular carcinoma | 20 | 256 | 4 +++ |
| Lung, NSC | LXFA 289 | adeno carcinoma, md | 18 | 326 | 12 ++ |
| | LXFA 526 | adeno carcinoma, pd | 9 | 860 | 2 +++ |
| | LXFL 1647 | large cell lung carcinoma | 7 | 604 | 5 +++ |
| | LXFL 529 | large cell lung carcinoma, ud | 15 | 456 | 0 +++ |
| Lung, SC | LXFS 615 | small cell lung carcinoma | 19 | 307 | 14 ++ |
| | LXFS 650 | small cell lung carcinoma, md | 11 | 334 | 0 +++ |
| Breast | MAXF 1322 | pap. adeno carcinoma, pd | 13 | 323 | 0 +++ |
| | MAXF 1384 | adeno carcinoma, pd | 20 | 448 | 4 +++ |
| | MAXF 401 | pap. adeno carcinoma, wd | 11 | 499 | 4 +++ |
| Melanoma | MEXF 1539 | Melanoma | 14 | 645 | 0 +++ |
| | MEXF 514 | melanotic melanoma | 18 | 573 | 4 +++ |
| | MEXF 989 | amelanotic melanoma | 18 | 591 | 0 +++ |
| Ovary | OVXF 550 | Carcinoma | 20 | 123 | 33 + |
| | OVXF 899 | pap. serous adeno carcinoma, md | 18 | 596 | 31 + |
| Pancreas | PAXF 736 | adeno carcinoma, pd | 18 | 351 | 5 +++ |
| Prostate | PRXF DU145 | adeno carcinoma, ud | 13 | 806 | 1 +++ |
| | PRXF MRIH1579 | adeno carcinoma | 14 | 495 | 7 +++ |
| Pleuramesothelioma | PXF 1118 | biphasic pleuramesothelioma | 20 | 387 | 2 +++ |
| Kidney | RXF 631 | hypernephroid adeno carcinoma, | 8 | 166 | 4 +++ |
| | | wd | | | |
| | RXF 944LX | hypernephroid carcinoma, clear | 7 | 650 | 1 +++ |
| | | cell | | | |
| Sarcoma | SXF 627 | pleomorphic rhabdomyosarcoma | 17 | 779 | 2 +++ |

^{a)}Mean value of respective experiment.
^{b)}5-FU at a concentration of 1.0 mg/ml
− (T/C > 50),
+ (30 ≦ T/C ≦ 50),
++ (10 < T/C < 30),
+++ (T/C ≦ 10).

TABLE 2
In vitro response rate towards Cytostatic
IN-VITRO EFFECT OF CYTOSTATIC IN HUMAN TUMOR XENOGRAFTS
Apr. 04, 2006

| TUMOR/ | | | | | | | | |
|---|---|---|---|---|---|---|---|---|
| PASSAGE | EXP. | COLONY | Test/Control (%) at Drug Concentration [%] | | | | | |
| NO. | NO. | CONTR. | .001 | .01 | .1 | 1. | 10. | 100. |
| BXF | | | | | | | | |
| 1218/14 | G033DM | 623 | 102 − | 97 − | 92 − | 0 +++ | 0 +++ | 0 +++ |
| CXF | | | | | | | | |
| 1103/9 | G018DM | 265 | 91 − | 78 − | 74 − | 20 ++ | 23 ++ | 22 ++ |
| 280/8 | F413AM | 403 | 92 − | 96 − | 70 − | 0 +++ | 6 +++ | 1 +++ |
| GXF | | | | | | | | |
| 1172/4 | G114FM | 579 | 94 − | 96 − | 97 − | 13 ++ | 8 +++ | 1 +++ |
| HNXF | | | | | | | | |
| 536/7 | G078CM | 140 | 85 − | 85 − | 99 − | 1 +++ | 4 +++ | 13 ++ |
| LIXF | | | | | | | | |
| 575/7 | G074DM | 234 | 98 − | 78 − | 85 − | 1 +++ | 2 +++ | 2 +++ |
| LXFA | | | | | | | | |
| 289/16 | G022DM | 292 | 91 − | 72 − | 93 − | 44 + | 51 − | 22 ++ |
| 526/9 | G014DM | 876 | 94 − | 91 − | 88 − | 1 +++ | 4 +++ | 0 +++ |
| LXFL | | | | | | | | |
| 1647/5 | G045DM | 639 | 91 − | 82 − | 85 − | 1 +++ | 4 +++ | 0 +++ |
| 529/7 | F414AM | 432 | 87 − | 64 − | 38 + | 0 +++ | 0 +++ | 0 +++ |
| LXFS | | | | | | | | |
| 615/12 | G081FM | 235 | 88 − | 59 − | 59 − | 0 +++ | 4 +++ | 6 +++ |
| 650/7 | G012DM | 334 | 26 ++ | 0 +++ | 0 +++ | 0 +++ | 0 +++ | 0 +++ |
| MAXF | | | | | | | | |
| 1322/6 | G009DM | 320 | 92 − | 72 − | 94 − | 0 +++ | 1 +++ | 0 +++ |
| 1384/13 | G092JM | 425 | 80 − | 72 − | 75 − | 6 +++ | 12 ++ | 5 +++ |
| 401/21 | G058DM | 428 | 97 − | 77 − | 47 + | 3 +++ | 9 +++ | 3 +++ |
| MEXF | | | | | | | | |
| 1539/10 | G086DM | 655 | 100 − | 89 − | 100 − | 0 +++ | 0 +++ | 0 +++ |
| 514/13 | G003AM | 485 | 87 − | 69 − | 68 − | 3 +++ | 9 +++ | 13 ++ |
| 989/11 | G002DM | 576 | 82 − | 0 +++ | 1 +++ | 0 +++ | 0 +++ | 0 +++ |
| OVXF | | | | | | | | |
| 550/14 | G109CM | 97 | 80 − | 72 − | 85 − | 79 − | 31 + | 25 ++ |
| 899/31 | G021DM | 531 | 100 − | 95 − | 84 − | 18 ++ | 21 ++ | 21 ++ |
| PAXF | | | | | | | | |
| 736/8 | G047DM | 265 | 102 − | 94 − | 86 − | 41 + | 15 ++ | 13 ++ |
| PRXF | | | | | | | | |
| DU145/4 | G073KM | 806 | 95 − | 80 − | 70 − | 2 +++ | 3 +++ | 8 +++ |
| MRIH1579 | G083AM | 326 | 86 − | 56 − | 47 + | 1 +++ | 2 +++ | 0 +++ |
| PXF | | | | | | | | |
| 1118/5 | G031DM | 319 | 102 − | 85 − | 90 − | 1 +++ | 2 +++ | 2 +++ |
| RXF | | | | | | | | |
| 631/13 | G023AM | 165 | 90 − | 108 − | 96 − | 1 +++ | 0 +++ | 0 +++ |
| 944LX | * (2) | 616 | 95 − | 80 − | 79 − | 1 +++ | 0 +++ | 0 +++ |
| SXF | | | | | | | | |
| 627/6 | G089GM | 764 | 86 − | 77 − | 95 − | 93 − | 29 ++ | 13 ++ |

| | | | | | | |
|---|---|---|---|---|---|---|
| Active (++, +++)/Total | 1/27 | 2/27 | 2/27 | 23/27 | 25/27 | 27/27 |
| | 4% | 7% | 7% | 85% | 93% | 100% |

AHS Hematopoietic Stem Cells;
AT Animal Tumor;
BXF Bladder Cancer Xenograft;
CEXF Cervix;
CNXF Central Nervous System
CXF Colorectal;
GXF Gastric;
HNXF Head and Neck;
LEXF Leukemia;
LXF Lung A adeno,
L large cell,
E epidermoid,
S small cell
LYXF Lymphoma;
MAXF Breast;
MEXF Melanoma;
OVXF Ovarian;
PAXF Pancreas;
PRXF Prostate;
PXF Pleuramesothelioma;
RXF Renal
SXF Sarcoma;
TXF Testicular;
UXF Uterine Body;
XF Miscellaneous
−, (T/C > 50);
+, (30 <= T/C <= 50);
++, (10 < T/C < 30);
+++, (T/C <= 10);
s, single plate result
B 2/evaluable experiments

TABLE 3
Effect of intramuscularly administered Cytostatic in NMRI nu/nu mice

| | | | | | Max. Med. |
|---|---|---|---|---|---|
| | Daily | Schedule | | Mortality n | BWL % |
| Therapy | Dose | [Day] | Route | (Day)¹ | (Day)² |
| Vehicle | 200 μL/ | 0-14 (H: 0 + 6) | im | 0/3 | 0.7 (3) |
| (0.9% | mouse | | | | |
| NaCl) | | | | | |
| Cytostatic | 200 μL/ | 0-14 (H: 0 + 6) | im | 0/4 | 1.7 (14) |
| | mouse | | | | |

n.r. = not relevant (no body weight loss observed)
¹Number of mice that died over total number of mice (days on which mice died)
²Day on which the minimum median body weight was recorded

TABLE 4
Necropsy Data
Vehicle control group

| | Mouse # | | |
|---|---|---|---|
| | #8740 | #7775 | #8490 |
| General condition | good | good | good |

| | | | | |
|---|---|---|---|---|
| Thorax | Heart | no abnormalities | Heart/lung | no abnormalities |
| | Lung | no abnormalities | complex with | no abnormalities |
| | Pleura | no abnormalities | deep-red bodies | no abnormalities |
| | | | above base of | |
| | | | heart in | |
| | | | mediastinum | |
| Abdomen | Intestine | no abnormalities | no abnormalities | no abnormalities |
| | Liver | no abnormalities | no abnormalities | no abnormalities |
| | Spleen | no abnormalities | no abnormalities | no abnormalities |
| | Kidney | no abnormalities | no abnormalities | no abnormalities |
| | Testes | no abnormalities | no abnormalities | no abnormalities |
| | Peritoneum | no abnormalities | no abnormalities | no abnormalities |
| | Others | no abnormalities | no abnormalities | no abnormalities |

Cytostatic-treated group

| | Mouse # | | | |
|---|---|---|---|---|
| | #6946 | #6957 | #6959 | #6960 |
| General condition | good | good | good | good |

| | | | | | |
|---|---|---|---|---|---|
| Thorax | Heart | no abnormalities | no abnormalities | no abnormalities | no abnormalities |
| | Lung | no abnormalities | no abnormalities | no abnormalities | no abnormalities |
| | Pleura | no abnormalities | no abnormalities | no abnormalities | no abnormalities |
| Abdomen | Intestine | no abnormalities | Small intestine | no abnormalities | no abnormalities |
| | | | empty | | |
| | Liver | no abnormalities | no abnormalities | no abnormalities | no abnormalities |
| | Spleen | no abnormalities | no abnormalities | no abnormalities | Marginally |
| | | | | | enlarged |
| | Kidney | no abnormalities | no abnormalities | no abnormalities | no abnormalities |
| | Testes | no abnormalities | no abnormalities | no abnormalities | no abnormalities |
| | Peritoneum | no abnormalities | no abnormalities | no abnormalities | no abnormalities |
| | Others | no abnormalities | adipose | adipose | adipose |

TABLE 5
Blood Cell Analysis (Large Blood Count)

| | | | | | Band | | | | |
|---|---|---|---|---|---|---|---|---|---|
| | | | Basophilic | Eosinophilic | Neutrophils/ | Segmented | | | Basophilic |
| | Mouse | Leukocytes | Granulocytes | Granulocytes | Stab | Neutrophils | Lymphocytes | Monocytes | Granulocytes |
| Group | Number | [G/I] | [%] | [%] | cells [%] | [%] | [%] | [%] | absolute |
| 1 | #8740 | 5.6 | 0.0 | 10 | 3 | 21 | 64 | 1 | 0 |
| 1 | #7775 | 9.7 | 0.0 | 2 | 0 | 6 | 90 | 2 | 0 |
| 1 | #8490 | x | 0.0 | 2 | 0 | 37 | 59 | 2 | x |
| Mean | | 7.65 | 0 | 4.7 | 1 | 21.3 | 71 | 1.7 | 0 |
| 2 | #6946 | 3.2 | 0.0 | 4 | 0 | 37 | 55 | 4 | 0 |
| 2 | #6957 | 11.3 | 0.0 | 10 | 2 | 31 | 55 | 2 | 0 |
| 2 | #6960 | 4.9 | 0.0 | 3 | 2 | 41 | 51 | 3 | 0 |
| 2 | #6959 | 5.2 | 0.0 | 13 | 0 | 31 | 52 | 3 | 0 |
| Mean | | 6.15 | 0.0 | 7.5 | 1 | 35 | 53 | 3 | 0 |

| | | | Eosinophilic | Segmented | | | | |
|---|---|---|---|---|---|---|---|---|
| | Mouse | Granulocytes | Neutrophils | Lymphocytes | Monocytes | Atypical | | |
| Group | Number | absolute | absolute | absolute | absolute | cells | Anisocytosis | Polychromasia |
| 1 | #8740 | 557 | 1170 | 3565 | 56 | 0 | + | ++ |
| 1 | #7775 | 193 | 580 | 8694 | 193 | 0 | + | ++ |
| 1 | #8490 | x | x | x | x | 0 | + | ++ |
| Mean | | 375 | 875 | 6130 | 125 | 0 | + | ++ |
| 2 | #6946 | 129 | 1191 | 1771 | 129 | 0 | + | + |
| 2 | #6957 | 1134 | 3515 | 6237 | 227 | 0 | + | + |
| 2 | #6960 | 148 | 2021 | 2514 | 148 | 0 | 0 | ++ |
| 2 | #6959 | 675 | 1609 | 2699 | 156 | 0 | 0 | ++ |
| Mean | | 522 | 2084 | 3305 | 165 | 0 | + | ++ |

Absolute cell numbers were determined using a blood cell counter.
Percentages were determined by microscopic evalution of stained blood smears.
x: absolute cell numbers could not be determined because of blood clotting.
#8490: 3 large atypical cells, mostly round central nucleus hemmed by large basophil plasma border

TABLE 6
In vitro activity of CC towards 4 human tumor cell lines (IC₅₀ values)

| Cell line | | IC₅₀ [%, v/v] | top* | bot* |
|---|---|---|---|---|
| LXFL | 529L | 0.307 | 94.9 | 6.37 |
| MAXF | 401NL | 0.127 | 93.2 | 2.49 |
| LXFA | 289L | 0.657 | 109 | 5.45 |
| OVXF | 899L | 0.329 | 117 | 11.7 |

IC50 values were calculated according non-linear regression using the analysis software GraphPad Prism ®, Prism 5 for windows, version 5.01 (GraphPad Software Inc., CA)
*top and bottom (bot) are the plateaus given in T/C (%) reflecting the maximum response (top) or the maximal level of inhibition (bot)

While all of the fundamental characteristics and features of the present invention have been described herein, with reference to particular embodiments thereof, a latitude of modification, various changes and substitutions are intended in the foregoing disclosure and it will be apparent that in some instances, some features of the invention will be employed without a corresponding use of other features without departing from the scope of the invention as set forth. It should be understood that such substitutions, modifications, and variations may be made by those skilled in the art without departing from the spirit or scope of the invention. Consequently, all such modifications and variations are included within the scope of the invention as defined by the following claims.

What is claimed is:

1. A composition comprised of an aldehyde suspended in a solution of a pharmaceutically acceptable salt in water.

2. The composition according to claim 1 wherein the aldehyde is formaldehyde.

3. The composition according to claim 1 wherein the salt is sodium chloride.

4. The composition according to claim 2 wherein the formaldehyde is suspended into the solution at a concentration between 0.00004% to 1.1% (v/v).

5. The composition according to claim 2 wherein the formaldehyde is suspended into the solution at a concentration between 0.00012% to 0.12% (v/v).

6. The composition according to claim 2 wherein the formaldehyde is suspended into the solution at a concentration between 0.00004% to 0.069% (v/v).

7. The composition according to claim 3 wherein the sodium chloride is at a concentration of 0.9%.

8. The composition according to claim 3 wherein the sodium chloride is at a concentration of 0.1% and 2.0%.

9. A method of preparing a pharmaceutical composition comprising:
suspending an aldehyde in a solution of a pharmaceutically acceptable salt in water.

10. The method according to claim 9 wherein the aldehyde is formaldehyde.

11. The method according to claim 9 wherein the salt is sodium chloride.

12. The method according to claim 10 wherein the formaldehyde is suspended in the solution at a concentration of between 0.00004% to 1.1%.

13. The method according to claim 11 wherein the sodium chloride is at a concentration of 0.9%

14. The method according to claim 9 wherein the pharmaceutical composition is for treating cancer or a cancerous growth.

15. Use of a composition comprising an aldehyde suspended in a solution of a pharmaceutically acceptable salt in water for treating cancer or a cancerous growth.

16. The use according to claim 15 wherein the aldehyde is formaldehyde.

17. The use according to claim 15 wherein the salt is sodium chloride.

18. The use according to claim 16 wherein the formaldehyde is suspended into the solution at a concentration between 0.00004% to 1.1% (v/v).

19. The use according to claim 16 wherein the formaldehyde is suspended into the solution at a concentration between 0.00012% to 0.12% (v/v).

20. The use according to claim 16 wherein the formaldehyde is suspended into the solution at a concentration between 0.00004% to 0.069% (v/v).

21. The use according to claim 17 wherein the sodium chloride is at a concentration of 0.9%.

22. The use according to claim 17 wherein the sodium chloride is at a concentration of 0.1% and 2.0%.
